(19) United States
(12) Patent Application Publication
(10) Pub. No.: US 2025/0121153 A1
(43) Pub. Date: Apr. 17, 2025

(54) NO DELIVERY APPARATUS COMPRISING AN ANALYSIS LINE WITH PIEZOELECTRIC PUMP

(71) Applicant: INOSYSTEMS, Antony (FR)

(72) Inventors: Thierry BOULANGER, Philadelphia, PA (US); Frederic MARCHAL, Wissous (FR); Mary SCHMITT, Bagneux (FR)

(21) Appl. No.: 18/911,826

(22) Filed: Oct. 10, 2024

(30) Foreign Application Priority Data
Oct. 11, 2023 (FR) 2310861

(51) Int. Cl. A61M 16/10; A61M 16/00; A61M 16/08

(52) U.S. Cl. CPC A61M 16/10; A61M 16/0003; A61M 16/024; A61M 16/0891; A61M 2016/0033; A61M 2016/102; A61M 2202/0007; A61M 2202/0208; A61M 2202/0275; A61M 2205/0211; A61M 2205/0294; A61M 2205/3327; A61M 2205/3334; A61M 2205/50

(57) ABSTRACT

The invention relates to an NO delivery apparatus (1) having a main gas circuit for conveying an NO-containing gas, in particular an NO/N₂ mixture, an analysis line (110) comprising NO/NO₂ measuring device(s) (120, 121) configured to perform NO and/or NO₂ concentration measurements within said analysis line (110), and a controller (15) connected to said NO/NO₂ measuring device (120, 121) and configured to process the NO and/or NO₂ concentration measurements performed by said NO/NO₂ measuring device (120, 121). In addition, the analysis line (110) comprises piezoelectric suction device (141) controlled by the controller (15).

### CROSS REFERENCE TO RELATED APPLICATIONS

This application claims the benefit of priority under 35 U.S.C. § 119 (a) and (b) to French Patent Application No. 2310861, filed Oct. 11, 2023, the entire contents of which are incorporated herein by reference.

### BACKGROUND

The invention relates to an NO delivery device or apparatus, i.e. an apparatus for supplying an NO-containing gas, in particular for connection between a source of gaseous NO, such as a pressurized cylinder of NO, and the ventilation circuit or patient circuit supplied with gas by a medical ventilator, which apparatus comprises gas analysis means intended to be fluidically connected to the patient circuit, typically an NO delivery apparatus comprising an analysis line with piezoelectric pump comprising a piezoelectric suction pump.

Nitric oxide or NO, when inhaled, dilates the pulmonary vessels and increases oxygenation by improving gas exchange. These properties are used to treat various medical conditions, as described in particular by EP-A-560928, EP-A-1516639 and US-A-10,201,564.

Usually, a small quantity of gaseous NO (i.e. a few ppm vol.), diluted in nitrogen (N₂), is diluted in a gas flow containing oxygen, such as an N₂/O₂ mixture or air, or even pure oxygen, which is conveyed through the patient circuit of a gas supply installation, and the final gas mixture thus obtained, containing NO and oxygen and nitrogen, is then inhaled by the patient.

To do this, the patient circuit is fluidically connected to an NO delivery device supplying the NO-based gas flow, and furthermore to a medical ventilator supplying the oxygen-based gas flow, as is described in US-A-5,558,083.

Since NO is a therapeutic agent effective at very low concentrations (i.e. a few ppmv or tens of ppmv), it is essential to ensure its correct dosage in the final NO/N₂/O₂ gas flow in order, if necessary, to be able to adapt the NO dosage according to the patient's condition, i.e. improvement or deterioration.

Conversely, it is also important to ensure that the final NO/N₂/O₂ gas flow does not contain toxic species, or contains only a negligible quantity of toxic species, in particular NO₂ resulting from oxidation of some of the NO by O₂.

Consequently, to guarantee increased patient safety, the NO delivery apparatus generally has a gas-sampling line for sampling a portion of the final gas mixture intended for the patient, typically gas samples of the order of 250 ml/min, within the respiratory circuit carrying the final NO/N₂/O₂ gas flow, as described by US2006/207594. These gas samples are then analysed within the NO delivery apparatus in order to check whether the composition of the final NO/N₂/O₂ gas flow corresponds to the desired composition, in particular to ensure that:
the NO concentration is as close as possible to the desired dosage, for example of the order of 10 to 20 ppmv, and
the NO₂ concentration is as low as possible, typically 0 to 5 ppmv, because NO₂ is a toxic gas that can cause serious injury to the patient, even at low concentration.

At present, the sampling of the gas from the patient circuit is carried out by means of a diaphragm pump, such as the pump of batch reference 2002 available from the Thomas company or the pump designated V/P 200 available from Xavitech.

Diaphragm pumps generally comprise two flexible membranes or diaphragms, which are mounted facing each other and move in oscillation, that is to say move toward and away from each other at a given frequency. During the phase in which the membranes move away from each other, a suction phenomenon occurs and a portion of the gas to be analysed fills the volume thus created and separating the two membranes, whereas, during the phase in which the membranes move toward each other, the same gas to be analysed is expelled from the pump. By controlling the frequency of these oscillations, typically between 0 and 50 Hz, it is possible to sample a “constant” flow rate of gas to be analysed, for example 250 ml/min.

However, in practice, the use of a diaphragm pump poses problems for several reasons.

Firstly, their mode of operation entails that the sampled flow rate, by its nature, oscillates around a mean value, for example 250 ml/min. These oscillations affect the performance of the ventilator, particularly its ability to detect inspirations by a newborn patient. Indeed, since newborn patients have a low respiratory drive, that is to say produce low inspiratory efforts and mobilize equally low inspiratory volumes of a few ml, the ventilators for this population use a proximal flow rate sensor, that is to say a sensor measuring the flow rates inhaled and exhaled by the patient directly at their mouth, for example at the outlet of the intubation tube. This flow rate sensor serves on the one hand to monitor and/or measure the flow rates inhaled by the patient during an inspiratory phase, but also to detect an onset of inspiration and to trigger the delivery of a volume in order to assist said patient.

However, the oscillations created by the process of gas sampling by the diaphragm pump are also found at the flow rate sensor, which disturbs the ventilator and causes what is called self-triggering; that is to say the incorrect assessment that the patient has initiated an inspiration, which results in the inappropriate administration of a volume to the patient. Such self-triggering introduces a detrimental asynchrony between the patient and the ventilator.

To remedy this, users have to “strengthen” the inspiration detection mechanism by making this detection less sensitive. However, this has the effect of requiring the patient to produce an additional inspiratory effort in order to be able to “trigger” the delivery of a volume by the ventilator.

Moreover, the oscillations created by the diaphragm pumps cause not inconsiderable noise pollution, because the frequencies of oscillation are in the audible spectrum of the patients. The noise created by such pumps is of the order of 50 decibels (dB), which has a considerable adverse impact on patients' sleep cycles and potentially affects patient recovery.

Moreover, WO2015127085, EP2522384 and US20170348503 disclose NO delivery apparatuses comprising an analysis line for the NO-based gas, making it possible to ensure that its composition does indeed correspond to the one desired and does not contain compounds harmful to the patient, such as NO₂. These apparatuses use conventional suction pumps.

Hence, a problem is to limit the oscillations in the sampling flow rate in order to optimize the detection of inspiration by the ventilator, and to limit the noise generated by the gas-sampling process.

### SUMMARY

A solution according to the invention concerns an NO delivery apparatus or device comprising:
a main gas circuit for conveying an NO-containing gas, in particular an NO/N₂ mixture,
an analysis line comprising NO/NO₂ measuring means configured to perform NO and/or NO₂ concentration measurements within said analysis line, and
control means connected to said NO/NO₂ measuring means and configured to process the NO and/or NO₂ concentration measurements performed by said NO/NO₂ measuring means.

Furthermore, according to the invention:
the analysis line of the apparatus comprises piezoelectric suction means (i.e. a device) controlled by the control means in order to suction gas at a given suction flow rate,
the control means are further configured to control electric field generating means, which are configured to generate an electric field at an excitation frequency of at least 20 kHz approximately,
the piezoelectric suction means comprise a piezoelectric pump comprising at least one piezoelectric material chosen from materials capable of deforming proportionally to an electric field applied to said piezoelectric material, and
said piezoelectric material is subjected to said electric field generated by the electric field generating means, so as to expand or retract under the effect of the electric field applied, generating a phenomenon of suction or expulsion of gas by the piezoelectric pump.

Depending on the embodiment considered, the apparatus of the invention can comprise one or more of the following features:
the control means are configured to control the piezoelectric suction means in order to suction gas at a given suction flow rate of less than or equal to 400 ml/min, preferably of at least 50 ml/min, preferably of between 100 and 350 ml/min, more preferably of between 200 and 300 ml/min.
the given suction flow rate is, for example, of the order of 250 ml/min.
the control means are configured to control electric field generating means configured to generate an electric field at an excitation frequency of between approximately 20 and 30 kHz.
the surface area of the piezoelectric membrane is at least 30 mm².
the NO/NO₂ measuring means comprise an NO sensor and an NO₂ sensor.
the piezoelectric suction means (or device) comprise a piezoelectric suction pump exploiting, during its operation, the piezoelectric dynamic effect resulting from the frequency excitation.
the piezoelectric pump comprises at least one piezoelectric material having a crystalline or ceramic structure.
the piezoelectric material is chosen from materials capable of deforming proportionally under the effect of the electric field applied thereto.
the piezoelectric material comprises at least one metal.
the piezoelectric material comprises lead zirconate titanate (PZT).
the piezoelectric membrane is supplied with electric current, in particular alternating current.
the electric field generation is obtained by dynamic variation of the applied electric current passing through the piezoelectric membrane.
the electric field generating means are controlled by the control means.
the electric field generating means comprise an electric current converter, also called an inverter.
the current converter is configured to convert a direct current to alternating current at a given frequency.
the control means control the value of the frequency of variation of the alternating current.
the electric field generating means are arranged in the apparatus.
the electric field generating means are supplied with electric current, in particular with direct current.
said at least one piezoelectric material is deposited in a layer, preferably in a thin layer, onto a metal substrate in order to form a piezoelectric membrane having a surface area of at least 30 mm².
the thickness of the layer of piezoelectric material, preferably a thin layer, is between a few tens and a few hundreds of μm.
the metal substrate comprises titanium or aluminium.
said at least one piezoelectric material has a disc shape or similar.
said at least one piezoelectric material is disposed in a closed enclosure comprising an inlet orifice or port and an outlet orifice or port.

In addition, depending on the embodiment considered, the NO delivery apparatus of the invention can comprise one or more of the following features:
the NO/NO₂ measuring means comprise a first metal oxide semiconductor (MOS) sensor configured to determine the NO concentration, and a second MOS sensor configured to determine the NO₂ concentration, or alternatively a combined MOS sensor configured to determine the NO and NO₂ concentrations.
each MOS sensor comprises a sensitive layer comprising at least one metal oxide, a resistive track subjected to electrical potential and a pair of electrodes, for example a metal oxide chosen from titanium dioxide (TiO₂) and tin dioxide (SnO₂).
according to another embodiment, the NO/NO₂ measuring means comprise electrochemical sensors.
it comprises electrical supply means for supplying electric current to the various elements or components of the apparatus that need this in order to operate, for example to the control means, to the sensors, to the pump, etc. Preferably, the electrical supply means comprise a connection to the mains (110/220V) and/or a rechargeable battery or other battery, and optionally a current converter.
the electrodes are electrically connected to the control means.
the control means are configured to determine a concentration of NO and/or NO₂.
the measuring means also comprise an O₂ sensor.
it also comprises a flow rate sensor arranged on the analysis line.
it comprises an information display, controlled by the control means, for displaying (at least) the concentrations, i.e. contents, of NO and NO₂, and possibly of O₂.
the information display comprises a screen, in particular the screen of a GUI.
the information display comprises a touch panel display.
the information display comprises a screen in colour or in black and white.
the information display is configured to display values of concentrations of NO and NO₂, and possibly O₂, which have been determined by the control means.
the main gas circuit comprises flow rate control means for controlling the flow rate of gas in the main gas circuit, preferably at least one solenoid valve.
the main gas circuit comprises one or more gas passages, such as gas conduits or the like.
the main gas circuit comprises at least one gas inlet through which an NO/N₂ mixture can enter the main circuit.
the main gas circuit comprises at least one gas outlet through which the NO/N₂ mixture can exit the main circuit.
the flow rate control means are controlled by the control means to allow or prevent any circulation of gas in the main gas circuit; in particular said at least one solenoid valve is controlled by the control means (i.e. controlled solenoid valve).
the main gas circuit further comprises pressure control means, in particular a gas expansion device.
the control means comprise at least one microprocessor.
said at least one microprocessor is arranged on at least one electronic board.
the control means comprise at least one microprocessor implementing at least one algorithm.
the NO delivery apparatus comprises a housing or outer shell.
the gas analysis line, the main gas circuit and the control means are arranged in the housing of the apparatus.

Furthermore, the invention also relates to an installation for administering gas to a patient, i.e. dedicated to supplying an NO-containing gas mixture to a patient, i.e. a person, in need thereof, comprising:
an NO delivery apparatus for supplying an NO-based gas according to the invention, such as an NO/N₂ mixture,
a medical ventilator for supplying an oxygen-based gas, such as air or an O₂/N₂ mixture,
a patient circuit to which the NO delivery apparatus and the medical ventilator are fluidically connected,
and a proximal flow rate sensor arranged on the patient circuit and electrically connected to the medical ventilator.

Depending on the embodiment considered, the gas supply installation of the invention can comprise one or more of the following features:
the medical ventilator is a respiratory assistance apparatus supplying the patient circuit with a respiratory gas containing oxygen, typically at least 20 vol % approximately of oxygen, preferably at least 20 vol % of oxygen, especially air or an N₂/O₂ mixture.
the NO delivery apparatus is supplied with NO, in particular an NO/N₂ mixture, by one or more gas sources containing gaseous NO.
the one or more gas sources contain an NO/N₂ mixture containing between 100 and 2500 ppm by volume of NO (ppmv), the remainder being nitrogen, preferably less than 1500 ppmv, the remainder being nitrogen, preferably less than 1000 ppmv, the remainder being nitrogen.
preferably, the gas source contains an NO/N₂ mixture containing from 250 to 900 ppm by volume of NO, the remainder being nitrogen, for example of the order of 800 ppm by volume of NO, the remainder being nitrogen.
the one or more gas sources is a pressurized gas cylinder.
the patient circuit comprises an inspiratory branch and an expiratory branch.
the medical ventilator, the main gas circuit and the analysis line of the NO delivery apparatus are fluidically connected to the inspiratory branch of the patient circuit.
the inspiratory branch and the expiratory branch are connected at a junction piece, such as a Y-piece.
the patient circuit, in particular the inspiratory branch, supplies a respiratory interface, such as a tracheal intubation tube or a breathing mask.
the proximal flow rate sensor is arranged on the patient circuit near the respiratory interface and/or the junction piece, i.e. Y-piece.
the proximal flow rate sensor is arranged between the junction piece and the respiratory interface.
a gas humidifier is arranged on the inspiratory branch, preferably downstream of the site of injection of the gaseous NO, i.e. of the NO/N₂ mixture, coming from the main gas circuit of the NO delivery apparatus.
the patient circuit is fluidically connected to an outlet port of the medical ventilator in order to collect and convey the oxygen-containing gas delivered by the medical ventilator, such as air or an N₂/O₂ mixture.
the one or more pressurized gas cylinders contain, when full, an NO/N₂ gas mixture at a pressure of at least 150 to 200 bar abs, or even at least 250 to 300 bar abs.
the patient circuit comprises conduits or hoses serving to convey gas.
the patient circuit comprises a main flow rate sensor electrically connected to the control means.
the analysis line of the NO delivery apparatus is fluidically connected to the inspiratory branch of the patient circuit at a gas-sampling site located downstream of the main flow rate sensor, preferably downstream of the gas humidifier.
the main gas circuit of the NO delivery apparatus is fluidically connected to the inspiratory branch of the patient circuit, at an injection site located between the main flow rate sensor and the gas-sampling site.
the main gas circuit of the NO delivery apparatus is fluidically connected to the inspiratory branch of the patient circuit via a gas injection line.
the analysis line of the NO delivery apparatus is fluidically connected to the inspiratory branch of the patient circuit via a gas-sampling line.
the gas analysis line of the NO delivery apparatus comprises an outlet port communicating with the ambient atmosphere.
the sampling line is connected to the patient circuit by means of a connection device, such as a T-piece, allowing gas samples to be taken from within the inspiratory branch of the patient circuit in order to check its composition, i.e. the NO and NO₂ concentrations.
the connection device is arranged downstream of the NO injection point, i.e. between the NO injection point and the patient interface, e.g. a tracheal intubation tube.
the medical ventilator supplies the patient circuit with a respiratory gas containing oxygen, typically at least 20 vol % approximately of oxygen, especially air or an N₂/O₂ mixture.
the NO delivery apparatus supplies the patient circuit with gaseous NO, in particular an NO/N₂ mixture, so as to dilute the gaseous NO (i.e. NO/N₂ mixture) in the oxygen-containing respiratory gas flow and obtain a final gas mixture to be administered to the patient.
the final gas mixture comprises essentially nitrogen (N₂), oxygen (O₂) at a concentration of at least 20 vol % approximately, and NO at a content of between 1 and 80 ppmv, typically of the order of 10 to 20 ppmv corresponding to a desired dosage, i.e. an NO/O₂/N₂ gas mixture.
the final gas mixture may comprise NO₂ species resulting from the oxidation of some of the NO.

### BRIEF DESCRIPTION OF THE DRAWINGS

The invention will now be better understood from the following detailed description, given as a non-limiting example, with reference to the appended figures, in which:

FIG. 1 is a partial schematic view of an installation for administering gas to a patient, comprising an NO delivery apparatus including a gas analyser with diaphragm pump according to the prior art.

FIG. 2 illustrates the flow rate oscillations generated by a diaphragm pump forming part of the gas analyser according to the prior art.

FIG. 3 is a schematic partial view of an installation for administering gas to a patient, comprising an NO delivery apparatus including a gas analyser with piezoelectric pump according to the present invention.

FIG. 4 shows the benefit of replacing the diaphragm pump of FIG. 1 with a piezoelectric pump according to the invention, as illustrated in FIG. 3.

### DETAILED DESCRIPTION OF PREFERRED EMBODIMENTS

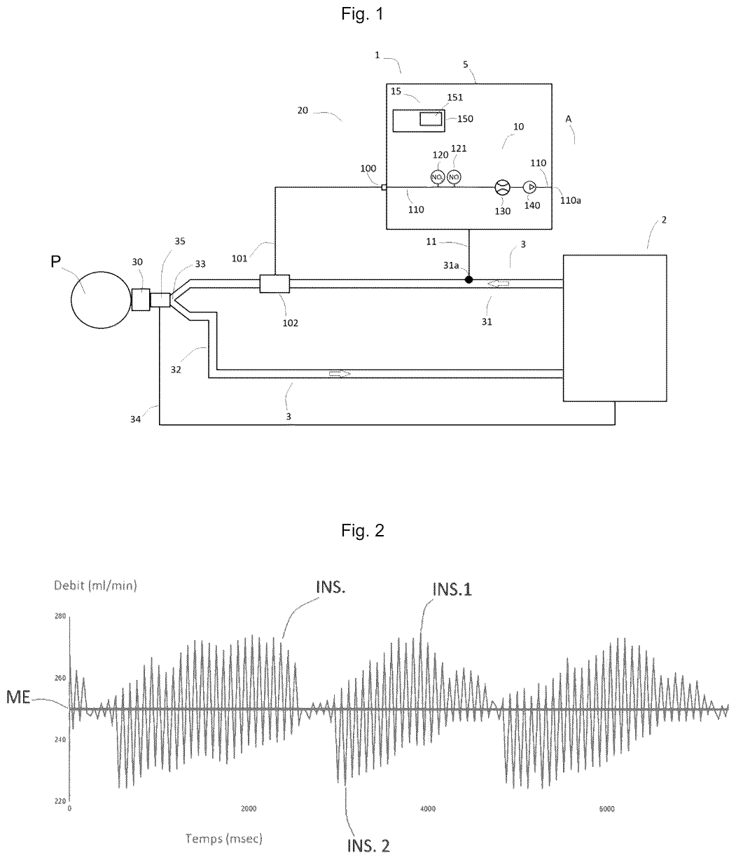

FIG. 1 schematically illustrates an embodiment of a gas delivery installation 20 for supplying gaseous NO to a patient P, comprising an NO delivery apparatus or device 1 for supplying an NO-based gas, and a medical ventilator 2, that is to say a respiratory assistance apparatus delivering a respiratory gas containing oxygen, typically at least 20% oxygen (O₂) approximately, generally at least 21% oxygen approximately.

Such an installation 20 makes it possible to deliver a final gas mixture containing NO at a desired concentration corresponding to a dosage fixed by an anaesthetist or the like, typically between 1 and 80 ppmv of NO (i.e. ppm by volume).

The final gas mixture is formed by mixing an NO-based flow of gas, such as an NO/N₂ mixture coming from the NO delivery apparatus 1, and a flow of respiratory gas containing oxygen, typically at least 20% oxygen (O₂) approximately, such as air or an O₂/N₂ mixture, coming from the medical ventilator 2.

The NO-based gas flow is injected (not shown) into a patient circuit 3, in particular into an inspiratory branch 31 of said patient circuit 3, which is also supplied with the oxygen-containing gas flow supplied by the medical ventilator 2.

The flow rate of the O₂-based gas flow circulating in the patient circuit 3 is measured by a main flow rate sensor (not shown) arranged on the patient circuit 3 downstream of the medical ventilator 2, typically on the inspiratory branch 31. The main flow rate sensor is electrically connected to the control means 15 of the NO delivery apparatus 1 in order to supply them with flow rate signals or measurements reflecting the flow rate of the oxygen-containing gas flow supplied by the medical ventilator 2 and circulating in the patient circuit 3 in the direction of the patient P, in particular in the upstream part of the inspiratory branch 31. The main flow rate sensor can be a flow rate sensor of the mass flowmeter type or pressure differential type, or similar.

The final gas mixture obtained by mixing the flows of NO/N₂ and of air or of an O₂/N₂ mixture for example is formed essentially of oxygen and nitrogen, and of NO at the desired dosage, typically between 1 and 80 ppmv of NO; it may contain unavoidable impurities, for example argon or the like. It is administered to the patient P, during their inspiratory phases, by means of a respiratory interface 3, for example a breathing mask, a tracheal intubation tube or any other suitable interface.

The gases exhaled by the patient during the expiratory phases are collected by an expiratory branch 32 of the patient circuit 3. The inspiratory branch 31 and expiratory branch 32 are fluidically connected to a junction piece 33, such as a Y-piece or the like, which is also connected to the respiratory interface 30.

The respiratory interface 30 makes it possible to deliver the gas to the patient P, in particular during their inspiratory phases, and to collect the gases exhaled by the patient P, in particular during their expiratory phases.

A proximal flow rate sensor 35 is also provided for measuring the flow rate administered to the patient and exhaled by the patient, which flow rate sensor is arranged at the respiratory interface 30 and/or at the junction piece 33, typically between them. The proximal flow rate sensor 35 may be of different technologies, such as a hot-wire sensor, and preferably adapted to the population of patients to be treated, i.e. neonates or adults. Here, an electric cable 34, connected to the medical ventilator 2, makes it possible to supply the proximal flow sensor 35 electrically. The instantaneous flow rate is measured in the medical ventilator 2 on the basis of the electrical information returned by the proximal flow rate sensor 35. Such technology is conventional.

The inspiratory branch 31 and expiratory branch 32 comprise conduits, channels, hoses, passages, tubes or similar, for example flexible polymer hoses that are able and configured to convey the gas flows.

During the operation of the installation 20, the gas flow circulating in the inspiratory branch 31 of the patient circuit 3, that is to say going from the mechanical ventilator 2 to the patient P, is inhaled by the patient P, while the gases exhaled by said patient P, i.e. enriched in CO₂, are conveyed via the expiratory branch 32 of the patient circuit 3 to the ventilator 2, where they are evacuated to the atmosphere via a venting orifice or the like.

Furthermore, the NO delivery apparatus 1 is of conventional architecture and operation as regards the supply of NO, typically an NO/N₂ mixture. It comprises (not shown) an internal main gas circuit for conveying an NO-containing gas, in particular one or more internal conveying lines for gas, typically an NO/N₂ mixture comprising from 100 to 2000 ppmv of NO (remainder N₂), typically less than 1000 ppmv.

Means for controlling the flow rate, such as a solenoid valve, and/or for controlling the pressure of the gas flow, such as a gas expansion device, are arranged on the main gas circuit. The flow rate control means are preferably governed by the control means 15 of the NO delivery apparatus 1 comprising one or more (micro)processors carried by one or more electronic cards 150, on the basis of the flow rate signals or measurements supplied by the main flow rate sensor. The main flow rate sensor is advantageously arranged on the patient circuit 3, typically the inspiratory branch 31, upstream of the injection site 31a of the inspiratory branch 31 of the patient circuit 3 where NO (i.e. NO/nitrogen mixture) is injected into the inspiratory branch 31 and mixes with the oxygen-containing gas flow coming from the medical ventilator 2.

The NO/N₂ mixture comprising, for example, from 100 to 2000 ppmv of NO (remainder N₂) generally comes from one or more gas sources, e.g. one or more cylinders of compressed gas (not shown) containing the NO/N₂ gas mixture at a pressure of up to 150 bar, or even 180 to 200 bar, or more.

The NO delivery device 1 makes it possible to supply/inject NO, that is to say the NO/nitrogen gas mixture, into the patient circuit 3 so that it mixes there with the oxygen-containing gas flow coming from the medical ventilator 2, as already explained.

More precisely, the NO/nitrogen mixture is conveyed via a gas injection line 11, such as a duct or the like, and the NO (i.e. the NO/nitrogen mixture) is injected at an injection site 31a of the inspiratory branch 31 of the patient circuit 3 so as to create there a mixture (i.e. a dilution) of the NO/nitrogen mixture flow with the oxygen-containing gas flow (i.e. >20 vol % approximately) coming from the medical ventilator 2, such as air or an O₂/N₂ mixture, and thus obtain the final NO/O₂/N₂ gas mixture.

The final gas mixture then administered to the patient P via the respiratory interface 30 therefore mainly comprises nitrogen (N₂), oxygen (O₂) in a content of at least 20 vol % approximately, and NO at a content of between 1 and 80 ppmv, typically of the order of 10 to 20 ppmv corresponding to a desired dosage.

Indeed, nitric oxide or NO, when inhaled, dilates the pulmonary vessels and increases oxygenation by improving gas exchange. These properties are used to treat various medical conditions such as persistent pulmonary hypertension of the newborn (PPHN), acute respiratory distress syndrome (ARDS) observed mainly in adults, or pulmonary hypertension (PH) in heart surgery, observed in adults or children, as described in particular by EP-A-560928, EP-A-1516639 and US-A-10,201,564.

The concentration of NO in the mixture administered to the patient corresponds to a dosage (i.e. a target concentration) determined by a physician or the like. In general, it is between 1 and 80 ppm by volume (ppmv), typically of the order of 10 to 20 ppmv, depending on the population treated, i.e. neonates, children, adolescents or adults, and on the disease to be treated.

However, on account of the presence of O₂ in the final NO/O₂/N₂ gas mixture, some of the NO present therein will oxidize and form toxic NO₂ species.

It is therefore essential to ensure that the concentration of NO in the final gas mixture does indeed correspond to the dosage desired by the physician and that, furthermore, the quantity of toxic NO₂ species is limited, typically less than a few ppmv, in general less than 0.5 ppmv.

To do this, the NO delivery device 1 of FIG. 1 incorporates gas analysis means 10, that is to say a gas analyser, making it possible to monitor, i.e. measure, the concentrations of NO and NO₂ in the final gas mixture, as explained below.

As has already been stated, the NO delivery apparatus 1 further comprises control means 15 comprising, for example, one or more electronic control boards 150 and a control unit 151 with (micro)processor(s), typically a microcontroller or the like. The control means 15 make it possible to adjust or control all the electromechanical elements of the NO delivery apparatus 1. More precisely, the electronic control board 150 preferably integrates the control unit 151, i.e. one or more microprocessors, and is configured to control and also to analyse the signals coming from the various components of the NO delivery apparatus 1, such as the pump, sensors, etc., including those from the gas analysis means 10.

The gas analysis means 10, that is to say the gas analyser, is arranged in the housing 5 or outer shell, for example made of polymer, of the NO delivery apparatus 1.

The gas analyser 10 comprises an inlet port 100 arranged on the outside of the housing 5 of the NO delivery apparatus 1 and fluidically connected to the inspiratory branch 31 of the patient circuit 3 via a gas-sampling line 101.

The sampling line 101 is connected to the patient circuit 3 by means of a suitable connection device 102, such as a T-piece or the like. allowing gas samples to be taken from within the inspiratory branch 31 of the patient circuit 3 in order to check its composition, in particular the NO and NO₂ concentrations.

The connection device 102 is arranged downstream of the NO injection point 31a, that is to say between the NO injection point 31a and the patient interface 30, e.g. a tracheal intubation tube.

The inlet port 100 of the gas analyser 10 fluidically communicates with a gas analysis line 110, which is arranged in the housing 5 and within which are arranged successively, starting from the inlet port 100, an NO₂ sensor 120 and an NO sensor 121, a flow rate sensor 130 and a gas suction device 140, such as a suction pump.

The gas analysis line 110 terminates at an outlet port 110a which is connected to the ambient atmosphere A and through which the gas is discharged into the atmosphere after having passed through the gas analysis line 110 and having been brought into contact with, in particular, the NO₂ sensor 120 and NO sensor 121.

The suction pump 140 makes it possible to circulate the gas by creating a flow of gas in the gas analysis line 110, as explained below.

The electrical power for the NO delivery apparatus 1, in particular for the control means 15, the gas analysis means 10 (i.e. the sensors 120, 121, 130) and the diaphragm pump 140 is provided by an electrical current source and/or electrical supply means (not shown), for example a connection to the mains current (110/220V), such as an electrical cord and connection socket, and/or one or more electric, preferably rechargeable, batteries, and/or a current transformer.

The gas flow to be analysed, which is suctioned by the pump and circulates through the gas analysis line 110, is brought into contact with the NO₂ sensor 120 and NO sensor 121, which will then measure the NO₂ and NO concentration of the gas flow and supply these measurements to the control means 15, which will then return them to the user, such as a physician or another caregiver, by ordering them to be displayed on an information display (not shown), such as a screen, of the graphical user interface (GUI) of the NO delivery apparatus 1.

The gas analysis line 110 can also comprise one or more other sensors, such as an oxygen sensor.

Of course, if necessary, the control means 15 can also process the NO and NO₂ measurements before ordering their display, in particular in order to perform compensation of these values so as to take account of environmental factors which can influence the measurements, such as atmospheric pressure, temperature and/or humidity, and to which the gas mixture may have been subjected.

According to the prior art, as has already been explained, the suction pump 140 is generally of the diaphragm type, also called a diaphragm pump. It is controlled by the control means 15 to suction, i.e. sample, some of the gas to be analysed coming from the patient circuit 3. Preferably, the flow rate circulating in the gas analysis line 110 is kept constant and equal to a given target flow rate, for example approximately 250 ml/min. This control of the diaphragm pump 140 is effected on the basis of the flow rate measurements performed by the flow rate sensor 130 of the analyser 10 and comprises adjustment, preferably permanently, of the control of the diaphragm pump 140 with a view to achieving the desired target flow rate.

In view of the intrinsic oscillatory nature of the diaphragm pump 140, as has been explained above, the control means 15, via in particular the control unit 151, can process the signal coming from the flow rate sensor 130 in order to “erase” the oscillations, by means of low-pass filters, time averages, etc., and allows said diaphragm pump 140 to reach and maintain the given target flow rate, for example 250 ml/min.

In all cases, with the pump 140 being of the diaphragm type, it induces oscillations around the target flow rate value, for example 250 ml/min.

Thus, FIG. 2 illustrates a flow rate versus time curve showing the oscillations which are generated by a diaphragm pump 140 used in the gas analysis line 110 of an NO delivery apparatus 1 according to the prior art, and which are measured by the flow rate sensor 130 of the gas analysis line 110.

It shows the instantaneous flow rate INS. derived from the instantaneous flow rate value transmitted by the flow rate sensor 130 and an averaged value ME derived from the signal processing performed by the control means 15, in particular via the control unit 151, which control the diaphragm pump 140 in order to reach and maintain the target sampling flow rate. It will be noted that this averaged value ME is in fact centred on the flow rate of 250 ml/min. However, it is clear that the instantaneous flow rate INS. is of an oscillating nature, alternating between values successively higher and lower than the averaged value ME.

For example, the maximum recorded flow rate of the instantaneous flow rate INS., INS. 1, is of the order of 275 ml/min, while the minimum recorded flow rate of the instantaneous flow rate INS., INS. 2, is of the order of 225 ml/min. This corresponds to flow rate variations of the order of 50 ml/min over time, which “interfere” with the measurement of the proximal flow rate 35 by propagating successively in the sampling line 101, the inspiratory branch 31 of the patient circuit 3 via the connection device 102, and the proximal flow rate sensor 35, giving rise to the abovementioned problems encountered with diaphragm pumps 140 according to the prior art.

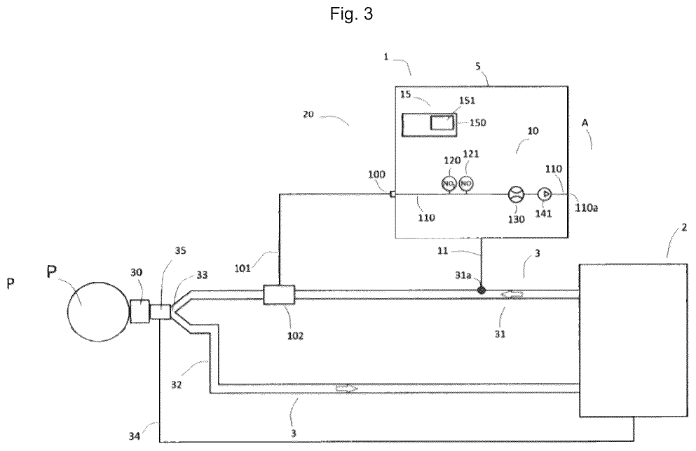

In order to solve these problems, according to the present invention, the diaphragm pump 140 according to the prior art in the NO delivery apparatus 1 of the installation 100 of FIG. 1 has been replaced by a piezoelectric pump 141, as is illustrated in FIG. 3. The rest of the installation 100 is unchanged and its operation is identical to that described with reference to FIG. 1.

More precisely, the piezoelectric pump 141 used according to the invention operates based on one or more piezoelectric materials, that is to say materials having piezoelectric properties, for example a structure of the crystalline or ceramic type, and responding to excitation by an electric field by deforming proportionally to the electric field to which they are subjected. For example, the piezoelectric material may be lead zirconate titanate, which can deform to a level of 0.1% of its dimensions under the effect of an electric field. Of course, the piezoelectric material may be any other suitable material.

The piezoelectric material is deposited in the form of one or more thin layers, of a few tens to hundreds of micrometers in thickness, onto a substrate of a metallic type, for example of titanium, aluminium or any other suitable substrate. A thin, i.e. fine, “membrane” is then obtained which is able to “oscillate” under the effect of a variable electric field.

In other words, a piezoelectric pump 141 may schematically comprise a surface, for example in the form of a disc or of another suitable shape, comprising a suitable substrate, for example a metal substrate, and one or more thin piezoelectric layers deposited thereon.

A piezoelectric assembly of this kind is placed in an enclosure, for example of circular cross section, forming a cavity provided with an inlet port and an outlet port. Moreover, electric field generation means are provided for generating a variable electric field (i.e. a conductive material) affecting the piezoelectric material.

The piezoelectric membrane is supplied with electric current, and the electric field generating means are controlled by the control means 15 to effect dynamic variations over time in the electric current supplied to the piezoelectric membrane. The electric field is then generated by the dynamic variations over time that are applied to the electric current passing through the piezoelectric assembly, that is to say the piezoelectric membrane, in particular an alternating electric current.

When the piezoelectric material expands or retracts, on account of the applied electric field, i.e. alternating electric current, a suction or expulsion phenomenon occurs via the inlet and outlet ports, respectively, of the piezoelectric pump 141. However, the small deformations of the piezoelectric material mobilize only a minute amount of gas, which then requires:
work of the piezoelectric membrane at high frequencies in order to significantly increase the number of oscillations of the piezoelectric material; and
dimensions of the piezoelectric membrane, typically of the surface of the piezoelectric membrane, and therefore of the cavity containing it, which are sufficient to mobilize “enough” gas by oscillation and to achieve the expected flow performance.

Hence, the excitation frequency of the piezoelectric material is preferably an ultrasonic frequency, that is to say at least 20 kHz, typically between 20 kHz and 30 kHz.

As is illustrated in FIG. 3, the control means 15 control the piezoelectric pump 141 in order to suction, i.e. sample, some of the gas to be analysed coming from the patient circuit 3 and passing through the sampling line 101, which is fluidically connected to the inspiratory branch 31.

Preferably, the control means 15 control the piezoelectric pump 141 so that the flow rate circulating in the gas analysis line 110 is kept constant and equal to a given target flow rate, typically of between 50 and 500 ml/min, for example of the order of approximately 250 ml/min. This control of the piezoelectric pump 141 is effected on the basis of the flow rate measurements performed by the flow rate sensor 130 and comprises adjustment, preferably permanently, of the control of the piezoelectric pump 141 with a view to achieving the desired target flow rate.

When the sampling line 101 is long, for example several metres, it may have a resistance to the flow of the sampled gas, which may affect the performance of the piezoelectric pump 141.

Consequently, at the abovementioned operating frequencies, the surface area of the piezoelectric membrane may be a factor to be taken into account in order to obtain the target flow rate, for example a target flow rate of 250 ml/min, considering moreover the resistance effected by the sampling line 101.

Tests carried out in the context of the invention have shown that the surface area of the piezoelectric membrane must be at least 30 mm², when operating at excitation frequencies of between 20 and 30 kHz.

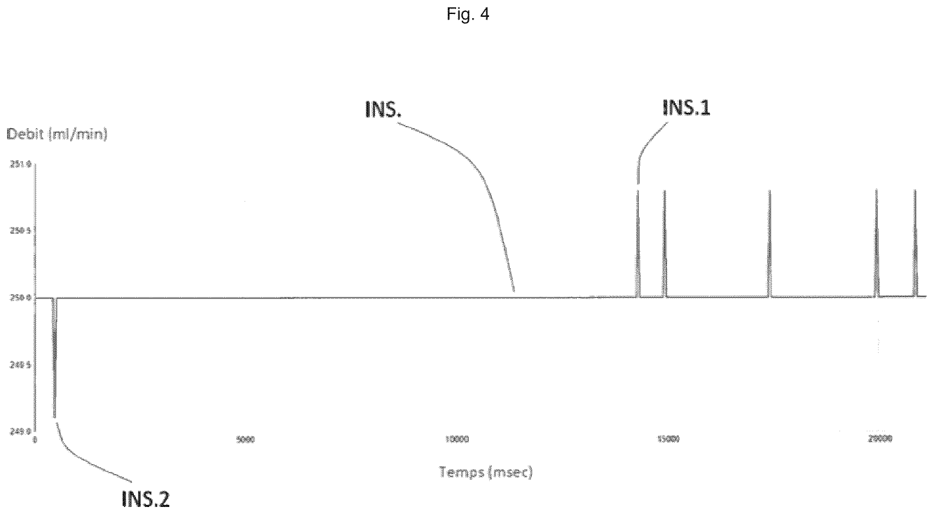

FIG. 4 shows the variations in the gas flow rate within the gas analysis line 110 when the gas is suctioned, according to the invention, with a piezoelectric pump 141 and the flow rate is measured by the flow rate sensor 130 of FIG. 3.

It shows the instantaneous flow rate INS. derived from the instantaneous value transmitted by the flow rate sensor 130, centred on the target flow rate, i.e. here 250 ml/min, via suitable control of the piezoelectric pump 141 by the control means 15, typically the control unit 151.

By virtue of using a piezoelectric pump 141 instead of the diaphragm pump 140 according to the prior art, the instantaneous flow rate INS. is perfectly smooth, that is to say has no oscillations or has extremely limited oscillations.

For example, over an acquisition of 20 seconds, only a few local maxima and minima INS. 1 and INS.2 occur, the value of which deviates only slightly from the target value, namely by only about 1 ml/min, that is to say more than 50 times lower than in the case of FIG. 2 where a diaphragm pump 140 according to the prior art has been used.

The advantage of using a piezoelectric pump 141 in the gas analysis line 110 of the ventilator 1 according to the invention will be appreciated. In fact, the variations in flow rate over time are small and do not interfere with the measurement of the proximal flow rate 35, which makes it possible to improve the sensitivity of detection of inspiratory effort of the medical ventilator 2, and thus to facilitate the inspiratory effort of the patients, especially when the patients are neonates.

In addition, the piezoelectric pump 141 also has the advantage of being completely silent, because the operating frequencies situated in the ultrasound range are inaudible to the human ear.

Generally, the gas administration installation 20 comprising the NO delivery apparatus 1 according to the invention is suitable for use in treating various pulmonary pathologies, such as persistent pulmonary hypertension of the newborn (PPHN), acute respiratory distress syndrome (ARDS) and/or pulmonary hypertension (PH) in heart surgery, namely in different patient populations, particularly neonates, adults, adolescents or children.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain the of within scope “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A Nitric Oxide (NO) delivery apparatus (1) comprising:
a main gas circuit for conveying an NO-containing gas,
an analysis line (110) comprising a NO/NO₂ measuring device (120, 121) configured to perform NO and/or NO₂ concentration measurements on the NO-containing gas within said analysis line (110), and
a controller (15) connected to said NO/NO₂ measuring device (120, 121) and configured to process the NO and/or NO₂ concentration measurements performed by said NO/NO₂ measuring device (120, 121),
characterized in that:
the analysis line (110) comprises piezoelectric suction device (141) controlled by the controller (15) configured to suction gas at a selected suction flow rate,
the controller (15) is further configured to control an electric field generator, which is configured to generate an electric field at an excitation frequency of at least 20 kHz,
the piezoelectric suction device (141) comprises a piezoelectric pump comprising at least one piezoelectric material chosen from materials capable of deforming proportionally to an electric field applied to said piezoelectric material, and
said piezoelectric material is configured to be subjected to said electric field generated by the electric field generator, so as to expand or retract under the effect of the electric field applied, thereby generating a phenomenon of suction or expulsion of gas by the piezoelectric pump.

2. The apparatus according to claim 1, characterized in that the piezoelectric material has a crystalline or ceramic structure.

3. The apparatus according to claim 1, characterized in that the given suction flow rate is less than 400 ml/min.

4. The apparatus according to claim 1, characterized in that the controller (15) is configured to control electric field generator to generate an electric field at an excitation frequency between 20 and 30 kHz.

5. The apparatus according to claim 1, characterized in that the NO/NO₂ measuring device (120, 121) comprises an NO sensor (121) and a separate NO₂ sensor (120).

6. The apparatus according to claim 1, characterized in that the piezoelectric material comprises at least one metal.

7. The apparatus according to claim 6, characterized in that the piezoelectric material comprises lead zirconate titanate (PZT).

8. The apparatus according to claim 1, characterized in that the piezoelectric suction device (141) comprise a piezoelectric suction pump configured to exploit, during its operation, the piezoelectric dynamic effect resulting from the frequency excitation.

9. The apparatus according to claim 1, characterized in that said at least one piezoelectric material is layered onto a metal substrate in order to form a piezoelectric membrane having a surface area of at least 30 mm².

10. The apparatus according to claim 1, characterized in that the electric field generator comprises an electric current converter.

11. The apparatus according to claim 10, characterized in that the electric current converter is supplied with direct electric current.

12. The apparatus according to claim 11, characterized in that the current converter is configured to convert the direct current to alternating current at a given frequency.

13. The apparatus according to claim 11, characterized in that the controller is configured to control the value of the frequency of variation of the alternating current of the electric current converter.

14. The apparatus according to claim 9, characterized in that the piezoelectric membrane is supplied with alternating electric current.

15. An installation (20) for administering gas to a patient (P), comprising:
an NO delivery apparatus (1) for supplying an NO-containing gas according to claim 1,
a medical ventilator (2) for supplying an oxygen-containing gas,
a patient circuit (3) to which the NO delivery apparatus (1) and the medical ventilator (2) are fluidically connected,
and a proximal flow rate sensor (35) arranged on the patient circuit (3) and electrically connected to the medical ventilator (2).
